(19) United States
(12) Patent Application Publication
(10) Pub. No.: US 2009/0112314 A1
(43) Pub. Date: Apr. 30, 2009

(54) Multi-focal intraocular lens with asymmetric point spread function

(76) Inventors: Edwin J. Sarver, Carbondale, IL (US); Donald R. Sanders, Oakbrook, IL (US)

Correspondence Address: MCHALE & SLAVIN, P.A., 2855 PGA BLVD, PALM BEACH GARDENS, FL, 33410, US

(21) Appl. No.: 11/924,296

(22) Filed: Oct. 25, 2007

(51) Int. Cl. A61F 2/16

(52) U.S. Cl. 623/6.17; 623/6.11

(57) ABSTRACT

The present invention describes a multi-focal intraocular lens for the human eye. The intraocular lens of the present invention provides improved vision quality over a range of object distances without producing glare or halos. It also provides non-symmetric, or nearly symmetric, optical zones about the lens optical axis.

### FIELD OF THE INVENTION

The invention generally relates to an implanted intraocular lens; particularly, to a presbyopic pseudophakic or phakic intraocular lens with multiple focal regions that restore a degree of accommodation permitting both near and far vision.

### BACKGROUND OF THE INVENTION

The normal human eye has two refracting elements: the cornea and the crystalline lens. For good vision, the powers and spacing of the cornea and crystalline lens and the distance between the crystalline lens and the retina must be such that the image of an object is brought into focus at the retina. If the powers of these refracting elements or the distances within the eye do not provide sharp focus at the retina, an optical correction to the eye must be made to provide the individual with sharp vision so a high quality of life can be maintained. If the optics of the eye causes the focus to be in front of the retina, the eye is said to be myopic or near sighted. If the optics cause the focus to be behind the retina, the eye is said to be hyperopic or far sighted. If the optics cause a sharp focus at the retina, the eye is said to be emmetropic.

In the normal operation of the eye, the crystalline lens can alter its power through a combination of changing shape and changing location. This ability of the crystalline lens to change its power is called accommodation, and it allows an individual eye to focus on near or distant objects. As individuals reach middle age they begin to lose this ability to accommodate. This loss in the ability to accommodate is called presbyopia and is a natural consequence of aging.

The correction of a myopic or hyperopic eye can be accomplished in a number of ways. The most common method is a spectacle lens or contact lens. Less common, but increasing in popularity are corneal surgery corrections such as laser in situ keratomileusis (LASIK), photo refractive keratectomy (PRK), LASEK, or implanting rings or other inlays into the cornea.

Another method to correct for myopia or hyperopia is the implantation of an intraocular lens (IOL). If the IOL is implanted with the crystalline lens still in the eye, it is called a phakic IOL (PIOL). If this PIOL is located in front of the iris it is referred to as an anterior chamber PIOL. If it is located behind the iris and in front of the crystalline lens, it is referred to as a posterior chamber PIOL. Other complications, e.g., cataracts, may require that the defective crystalline lens be removed from the ocular system and a synthetic lens referred to as a pseudophakic intraocular lens be put in its place.

The monofocal PIOLs provide the myopic or hyperopic subject with vision correction for a single viewing distance and rely on the accommodation of the crystalline lens to adjust focus as the object distance decreases. If the subject is presbyopic so that the crystalline lens can no longer provide this focus change, some other means must be used to provide this range of focus or the range of adequate vision will be limited.

In addition to the optical system of the eye not being able to focus the light from a distant object onto the retina, the eye's focusing error may not be the same for each meridian of the eye. For example, the focusing error in the horizontal meridian could be −2 diopters (D), and in the vertical meridian it could be −4 D. In this case, the eye is said to have 2 D of astigmatism. The correction of this astigmatic error is often required to provide acceptable vision quality.

One way to provide a presbyopic patient with the ability to focus on near and distance objects (and essentially restore a degree of accommodation) is to provide an optic with multiple focal regions such as is provided by a bi-focal spectacle lens. This is typically done with annular regions in the IOL but can be done in non-annular regions. For example, in the U.S. Pat. No. 6,797,003 to Blake et al., discussed further below, the posterior surface is essentially spherical while the anterior surface has three sectors. The upper sector is essentially spherical and extends to the midsection of the disk. The center sector, adjacent to the upper sector, extends therefrom to the lower quarter of the disk and is formed of an aspherical sector of decreasing radius of curvature. The lower sector is also essentially spherical. The design provides for a continuously varying object distance, thus providing both near and far vision.

A problem with these types of IOLs in general is their propensity to produce a glare and halos in the patient's field of vision. It is believed that these problems are caused by the shape of individual refractive zones and the transition zones between the annular regions which direct unwanted light to specific regions on the retina. If this light is of sufficient power, the patient will perceive it as an artifact.

What has been heretofore lacking in the prior art is a presbyopic phakic IOL or presbyopic pseudophakic IOL that can provide good vision quality over a range of object distances and does not suffer from the same level of glare and halos that are visible in IOLs. This is accomplished by having optical zones which are not symmetric (or nearly symmetric) about the lens optical axis and steering the resulting asymmetric stray light in opposite directions (e.g., up and down or left and right) as the lens is implanted into the left and right eyes. The brain's higher level vision processing will tend to cancel the stray light aberrations between the two views and thus provide improved vision over traditional multi-focal IOLs. Also, if the eye is astigmatic, the IOL will incorporate an astigmatic correction.

### DESCRIPTION OF THE PRIOR ART

Numerous patents have been directed toward accommodating intraocular lenses for providing improved vision. For example, U.S. Pat. No. 6,797,003 to Blake et al., discloses an optical power surface, which may have multiple radii portions or aspherical portions, as well as spherical portions, intended to replace the crystalline lens of a patient's eye, in particular after a cataract extraction. Such an aspheric soft lens is molded in a coined mold.

U.S. Pat. No. 5,507,806 to Blake, discloses an improved multi-faceted intraocular lens with a main optical element having a plurality of optical elements. The flexible, thin multi-faceted intraocular lens is made of an optical-grade soft biocompatible material, or a silicone material. The thin, flat, multi-faceted intraocular lens may enable implantation of the lens through an intraocular lens injector having an injection tube with a diameter of approximately 1 mm to 4 mm. The plurality of optical elements each may have the same or differing diopter powers. Additionally, the plurality of optical elements may be aligned to form a multi-focal lens. Further, the optical elements each may be selected from a group consisting of toric elements, aspheric elements, and spherical elements depending upon the type of correction desired. Lastly, the multi-faceted intraocular lens may be effective in the treatment of age-related macular degeneration. This method is primarily concerned with placing multiple copies of an image on the retina in an attempt to treat age-related macular degeneration and other retinal anomalies. When configured to provide a multi-focal optic (two distances brought into focus at the same retinal point), the elements are arranged in annular ring(s) which will cause the same undesirable halo patterns as those provided by other annular multi-focal IOLs.

The problem with the aforementioned prior art IOLs are caused by the arrangement and shape of the focal regions and the transition zones between the annular regions, like the three-sector design in the '003 patent to Blake et al., which focuses unwanted light to specific region s on the retina, causing the patient to perceive the light as an artifact or as a blinding halo around the patient's field of vision. None of the aforementioned prior art have effectively addressed common problems found in IOLs, including visual aberrations and astigmatic error, which can have a negative impact on overall image quality. Also, other than monovision (correcting one eye for distance vision and the other eye for near vision) the present method of steering stray light aberrations in different directions between the two eyes is the only method that specifically employs both eyes and higher level vision processing by the brain to reduce typical multi-focal stray light artifacts.

### SUMMARY OF THE INVENTION

The instant invention is related to both presbyopic phakic and pseudophakic intraocular lenses that provide improved vision quality over a range of object distances. This is accomplished by having optical zones which are not symmetric (or nearly symmetric) about the lens optical axis and implanting the IOLs in the left and right eyes so that the asymmetric point spread functions are oriented in opposite directions.

In a particular embodiment, the invention relates to an intraocular lens, or a pair of such intraocular lenses, which may be phakic or pseudophakic, for treatment of an eye, or eyes, of a presbyopic patient, and include an optic body sized and configured to be received in an eye ( or eyes) of a presbyopic patient, said optic body including an anterior wall with an anterior optical center and a posterior wall with a posterior optical center, and having a lens optical axis intersecting the anterior wall at the anterior optical center and the posterior wall at the posterior optical center, and having optical zones which are not symmetric about the lens optical axis, wherein said lens construction produces an asymmetric point spread function which enables any resulting asymmetric stray light to be steered in a predetermined direction. When used for a pair of eyes, the lenses are constructed and arranged such that they include a left eye lens and a right eye lens, each lens having an optic body sized and configured to be received, respectively, in a left or right eye of a presbyopic patient, wherein each said lens construction produces an asymmetric point spread function which enables any resulting asymmetric stray light to be steered in a direction opposite to that of the other member of said pair of lenses, thereby enabling stray light aberrations to be canceled as a result of the patient's higher vision brain processing, and thereby providing improved vision over traditional multi-focal intraocular lenses.

It is an objective of the present invention to teach an intraocular lens designed for a specific individual's eye, that is, optimized for physiological conditions (e.g., pupil diameter) and visual preferences (e.g., distance clarity verses near clarity).

It is therefore an objective of the instant invention to provide an IOL that may incorporate a correction for simple defocus and/or astigmatism.

These and other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A illustrates the anterior view of a preferred optical design of an IOL of the present invention;

FIG. 1B illustrates the posterior view of the IOL of FIG. 1A;

FIG. 2 illustrates another embodiment of the present invention where the surface of the IOL is partitioned into four regions.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

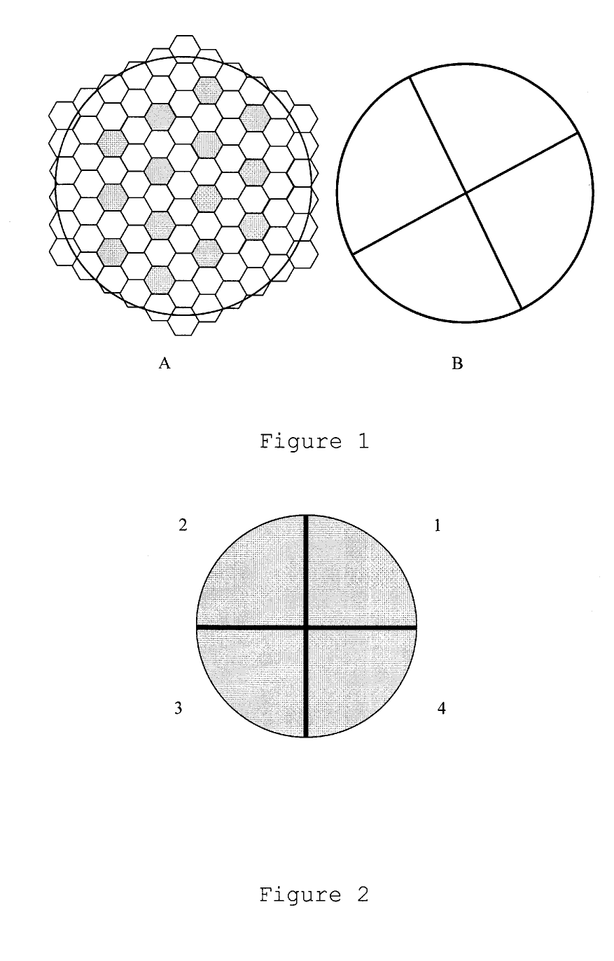

The preferred basic optical design of the IOL is illustrated in FIGS. 1A and 1B. FIG. 1A represents the anterior surface (the surface facing the cornea) of the IOL and FIG. 1B represents the posterior surface (the surface facing the crystalline lens) of the IOL.

The anterior surface is composed of individual regions of one optical power (the white hexagons) or another (the gray hexagons). The set of white hexagons represents a given focal power, for example for distance vision. The set of gray hexagons represents a given focal power, for example for near vision. Although the anterior surface in FIG. 1A is illustrated for two focal distances, any number of sets of focal distances could be added. The size and shape of the regions could be adjusted and the optic within each region could be aspheric to reduce overall aberrations. The individual hexagon assignment to a given focal distance set could be some regular pattern as illustrated in FIG. 1A or it could be random. Each hexagon could also represent a multi-focal region where the one half of the region provides one focusing power and the other half provides another focusing power. The manner in which the region is divided to provide the two powers is selected to distribute stray light on the retina to avoid typical halo patterns.

It should also be noted that the number of hexagons for the distance vision set is about 4 times that for the near vision set. This allows the relative importance of the distance and near vision sets to be adjusted based on any number of optimization criteria. Such optimization criteria could be based upon preference for distance clarity versus near clarity or could include physiologic conditions such as pupil size (diameter) due to illumination or accommodative demand. The optimization criteria could also include details such as the Stiles-Crawford effect.

The regions need not be hexagonal shaped. Squares or other shapes including random shapes may be equally useful. In addition, the regions need not be the same size. The regions could be realized using shape differences or differences in index of refraction. The total zone in which the regions are located could be limited to a given area of the surface such as the center of the lens or the periphery.

FIG. 1B illustrates that the back surface of the IOL could include a basic optical power and astigmatism. The back surface could also be aspheric to control overall aberrations. It is contemplated herein that the shapes of the anterior and posterior surfaces could be adjusted to account for astigmatism.

The use of common optical design principles known to those skilled in the art of IOL design can be used to determine the lens powers, surface radii, center thickness, and any other parameters described in the following discussion concerning the anterior and posterior surfaces of the multi-focal IOL optic.

A second partitioning called the radial sections partitioning is illustrated in FIG. 2. Each radial section represents a discrete optical power. In FIG. 2, the surface of the IOL is sectioned into radial regions and a constant power is applied to a region. The number of regions could be 1, 2, 3, 4 and so on (only four regions are shown in FIG. 2). The power profile between the regions could be discrete, discrete with blend regions, or continuously varying.

The radial sections could be complete or other zones such as a central zone could be added. A combination of radial sections (FIG. 2) and hexagonal shaped regions (FIGS. 1A) could be made on either or both the anterior and posterior surfaces of the IOL. The same optical principles could be applied to phakic as well as aphakic IOLs, contact lenses, spectacle lenses, corneal surgery (such a LASIK, LASEK, PRK), and corneal implants. These optical principles include the strategy of intentionally producing an asymmetric point spread function and steering the direction of the asymmetric point spread functions in the left and right eyes so the brain's higher level vision processing will tend to cancel the stray light aberration between the two views. Each radial section could have an aspheric profile. The individual shapes could be adjusted to control the overall aberrations of the IOL or the individual eye in which it is to be placed. The sectors need not be of equal angle.

The mathematical representation of the optical surface will typically be via some type of surface such as a spline (B-spline, etc), Fourier expansion, or Zernike polynomial expansion.

In addition to the target distance focus correction provided by the focal zones, if the patient has significant astigmatism, an astigmatic posterior surface is provided to correct this aberration.

For various optical designs above, the orientation and design of the regions in total should produce an asymmetric point spread function at a focal plane so that if rotated (or reflected) the resulting image from two such IOLs implanted in the left and right eyes would tend to cancel.

In addition to the aforementioned embodiments, the following extensions are further contemplated:

1. The anterior and posterior surfaces described above could be reversed, that is, the astigmatic power could be placed on the anterior surface and the focal zones could be placed on the posterior surface.

2. The astigmatic power and the focal zones could be incorporated into both surfaces either equally or by some fraction between the two surfaces.

3. The optical zones could be incorporated into the astigmatic posterior surface and the anterior surface could be spherical.

4. Aspheric surfaces or zones could be utilized to reduce aberrations of the lens.

5. The design of the optic could be such that a nonsymmetric point spread function could be produced by other means such as a diffractive optic or an optic created by altering the profile of refractive index inside the optic.

6. The design of the lens could be such that the resulting point spread function is symmetric, but has a smooth response outside of the central peak. That is, the stray light from the out of focus regions do not form sharp boundaries in the point spread function plane.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An intraocular lens for treatment of an eye of a presbyopic patient comprising:
an optic body sized and configured to be received in an eye of a presbyopic patient, said optic body including an anterior wall with an anterior optical center and a posterior wall with a posterior optical center, and having a lens optical axis intersecting the anterior wall at the anterior optical center and the posterior wall at the posterior optical center, and having optical zones which are not symmetric about the lens optical axis, wherein said lens construction produces an asymmetric point spread function which enables any resulting asymmetric stray light to be steered in a predetermined direction.

2. A pair of intraocular lenses for treatment of the eyes of a presbyopic patient comprising:
a pair of lenses including a left eye lens and a right eye lens, each lens having an optic body sized and configured to be received, respectively, in a left or right eye of a presbyopic patient, each said lens having an optic body including an anterior wall with an anterior optical center and a posterior wall with a posterior optical center, and having a lens optical axis intersecting the anterior wall at the anterior optical center and the posterior wall at the posterior optical center, and having optical zones which are not symmetric about the lens optical axis, wherein each said lens construction produces an asymmetric point spread function which enables any resulting asymmetric stray light to be steered in a direction opposite to that of the other member of said pair of lenses;
whereby stray light aberrations are canceled as a result of the patient's higher vision processing, thereby providing improved vision over traditional multi-focal intraocular lenses.

3. The intraocular lens of claim 1 wherein the lens is optimized for physiological conditions including pupil diameter and visual preferences;
thereby providing for distance clarity verses near clarity for a specific individual's eye.

4. The intraocular lenses of claim 2 wherein each said lens is optimized for physiological conditions including pupil diameter and visual preferences;
thereby providing for distance clarity verses near clarity for a specific individual's eyes.

5. The intraocular lens of claim 1 further including an astigmatic correction.

6. The intraocular lenses of claim 2 further including an astigmatic correction.

7. The intraocular lens of claim 1 wherein one wall of said lens includes a plurality of individual regions, each individual region having a distinct optical power, and being constructed and arranged aspherically to reduce overall aberrations;
whereby said lens is optimized based upon patient preference for near, middle or distance clarity.

8. The intraocular lenses of claim 2 wherein one wall of each said lens includes a plurality of individual regions, each individual region having a distinct optical power, and being constructed and arranged aspherically to reduce overall aberrations;
whereby each said lens is optimized based upon patient preference for near, middle or distance clarity.

9. The intraocular lens of claim 7 further including an astigmatic correction.

10. The intraocular lens of claim 8 further including an astigmatic correction.

11. The intraocular lens of claim 7 wherein said individual regions are partitioned as radial sections.

12. The intraocular lens of claim 8 wherein said individual regions are partitioned as radial sections.

13. The intraocular lens of claim 7 wherein said individual regions are partitioned as polygonal sections, radial sections, or a combination thereof.

14. The intraocular lenses of claim 8 wherein each said lenses individual regions are partitioned as polygonal sections, radial sections, or a combination thereof.

15. The intraocular lens of claim 7 wherein the astigmatic correction and individual optical power regions are incorporated into both the anterior and posterior walls either equally or by some fraction between the two walls.

16. The intraocular lenses of claim 8 wherein the astigmatic correction and individual optical power regions of each lens are incorporated into both the anterior and posterior walls either equally or by some fraction between the two walls.

17. The intraocular lens of claim 1 wherein a nonsymmetric point function is produced by means of a diffractive optic, or an optic created by altering the profile of refractive index inside the optic.

18. The intraocular lenses of claim 2 wherein a nonsymmetric point function is produced, in at least one of said lenses, by means of a diffractive optic, or an optic created by altering the profile of refractive index inside the optic.
